(19) United States
(12) Patent Application Publication
(10) Pub. No.: US 2015/0209165 A1
(43) Pub. Date: Jul. 30, 2015

(54) VASCULAR AND BODILY DUCT TREATMENT DEVICES AND METHODS

(71) Applicant: Concentric Medical, Inc., Mountain View, CA (US)

(72) Inventors: Ryan M. Grandfield, San Jose, CA (US); Scott D. Wilson, Redwood City, CA (US); Aleksandr Leynov, Walnut Creek, CA (US); Masao Drexel, Santa Cruz, CA (US)

(73) Assignee: Concentric Medical, Inc., Mountain View, CA (US)

(21) Appl. No.: 14/607,643

(22) Filed: Jan. 28, 2015

Related U.S. Application Data
(60) Provisional application No. 61/933,113, filed on Jan. 29, 2014.

(51) Int. Cl. A61F 2/88; A61F 2/04; A61F 2/844

(52) U.S. Cl. CPC A61F 2/88; A61F 2/844; A61F 2/04

(57) ABSTRACT

Treatment devices having a self-expandable member with one or more of a proximal end portion, a main body portion and a distal portion. According to some implementations a self-expandable member is provided with proximal end portion comprising a peripheral rail having a first substantially straight segment and a second substantially straight segment that extends distally to the first substantially straight segment with the angular orientation of the second substantially straight segment being different than the angular orientation of the first substantially straight segment, the angular orientation of the second substantially straight segment being similar to a helix angle of at least some of the cell structures in the main body portion. According to other implementations an expandable member is provided that includes a plurality of distal-most cell structures with each of the plurality of distal-most cell structures having a pair of distal-most struts that at least partially form an end segment of the respective distal-most cell structures, the distal-most struts having a proximal region and a distal region with at least some of the proximal regions having a width and/or thickness dimension less than the width and/or dimension of the respective distal regions.

### TECHNICAL FIELD

This application relates to devices and methods for treating the vasculature and other ducts within the body.

### BACKGROUND

Self-expanding prostheses, such as stents, covered stents, vascular grafts, flow diverters, and the like have been developed to treat ducts within the body. Many of the prostheses have been developed to treat blockages within the vasculature and also aneurysms that occur in the brain. What are needed are improved treatment methods and devices for treating the vasculature and other body ducts, such as, for example, aneurysms, stenoses, embolic obstructions, and the like.

### SUMMARY OF THE DISCLOSURE

In accordance with one implementation a vascular or bodily duct treatment device is provided that comprises an elongate self-expandable member expandable from a first delivery position to a second placement position, in the first delivery position the expandable member being in an unexpanded position and having a nominal first diameter and in the second position the expandable member being in a radially expanded position and having a second nominal diameter greater than the first nominal diameter, the expandable member comprising a plurality of diagonally disposed cell structures, the expandable member having a proximal end portion and a substantially cylindrical main body portion disposed distal to the proximal end portion, the cell structures in the main body portion extending circumferentially around a longitudinal axis of the expandable member, the cell structures in the proximal end portion extending less than circumferentially around the longitudinal axis of the expandable member to form first and second peripheral rails, the first peripheral rail comprising a first substantially straight segment that originates at or near a proximal end of the expandable member and a second substantially straight segment that extends from the first substantially straight rail segment to a location at or near the substantially cylindrical main body portion, the angular orientation of the second substantially straight segment being different than the angular orientation of the first substantially straight segment, the angular orientation of the second substantially straight segment being similar to a helix angle of the cell structures in the main body portion.

In accordance with one implementation a vascular or bodily duct treatment device is provided that comprises an elongate self-expandable member expandable from a first delivery position to a second placement position, in the first delivery position the expandable member being in an unexpanded position and having a nominal first diameter and in the second position the expandable member being in a radially expanded position and having a second nominal diameter greater than the first nominal diameter, the expandable member comprising a plurality of diagonally disposed cell structures, the expandable member having a proximal end portion, a substantially cylindrical main body portion disposed distal to the proximal end portion and a distal portion disposed distal to the substantially cylindrical main body portion, the cell structures in the substantially cylindrical main body portion and distal portion extending circumferentially around a longitudinal axis of the expandable member, the cell structures in the proximal end portion extending less than circumferentially around the longitudinal axis of the expandable member, the distal portion comprising a plurality of distal-most cell structures with each of the plurality of distal-most cell structures comprising a pair of distal-most struts that at least partially form an end segment of the respective distal-most cell structures, the distal-most struts comprising a proximal region and a distal region with at least some of the proximal regions having a width and/or thickness dimension less than the width and/or dimension of the respective distal regions.

These and a host of other features associated with improved vascular and bodily duct treatment devices are disclosed herein.

### BRIEF DESCRIPTION OF THE DRAWINGS

FIG. 1A illustrates a two-dimensional plane view of an expandable member of a treatment device according to one implementation.

FIG. 1B is an isometric view of the expandable member illustrated in FIG. 1A.

FIG. 1C again illustrates the expandable member of FIG. 1A.

FIG. 1D illustrates an enlarged view of the proximal end of the expandable member of FIG. 1A.

FIG. 1E illustrates an enlarged view of the distal end of the expandable member of FIG. 1A.

FIG. 1F illustrates an enlarged view of the distal portion of a distal-most cell structure in the expandable member of FIG. 1A.

FIG. 2 illustrates a two-dimensional plane view of an expandable member of a treatment device according to another implementation.

FIG. 3 illustrates a distal portion of an expandable member according to one implementation.

FIG. 4 illustrates a two-dimensional plane view of an expandable member of a treatment device according to another implementation.

FIG. 5 illustrates a two-dimensional plane view of an expandable member of a treatment device according to another implementation.

FIG. 6 illustrates the expandable member of FIG. 2 having a plurality of radiopaque wires wound about selective struts.

FIG. 7 illustrates a two-dimensional plane view of an expandable member of a treatment device according to another implementation.

FIG. 8A illustrates a two-dimensional plane view of an expandable member of a treatment device according to another implementation.

FIG. 8B illustrates the expandable member of FIG. 8A having a plurality of wires wound about selective struts.

### DETAILED DESCRIPTION

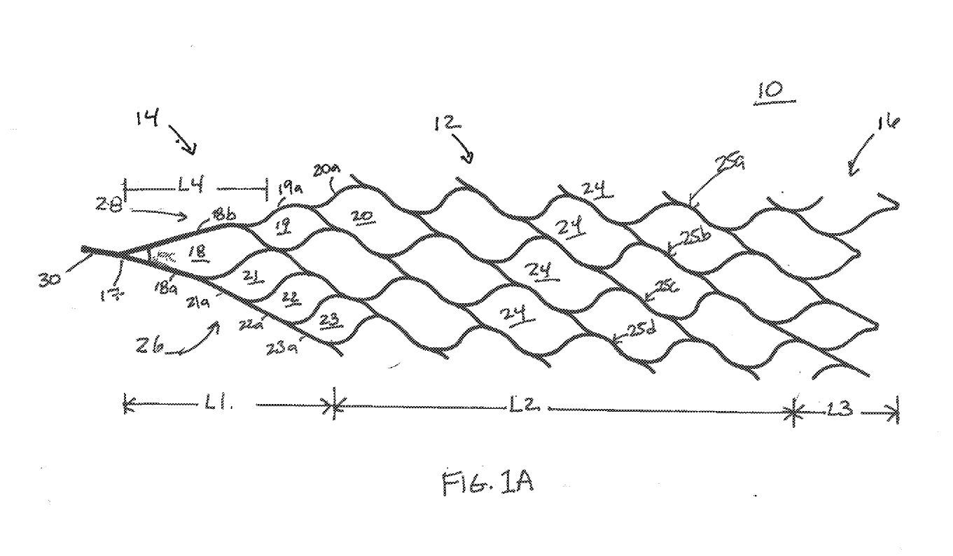

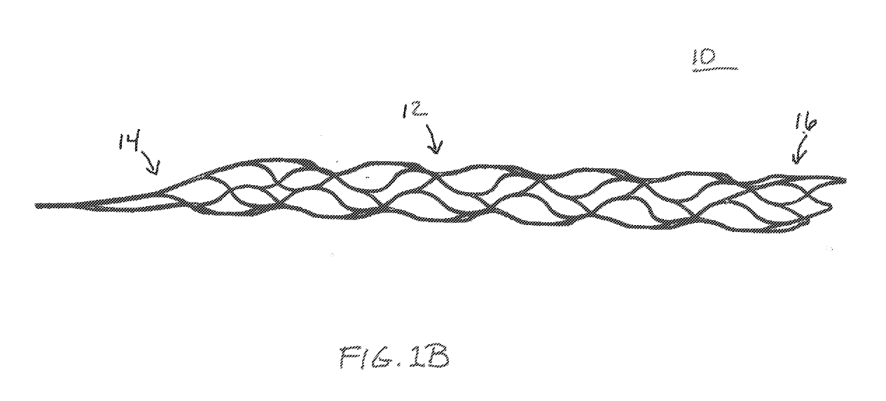

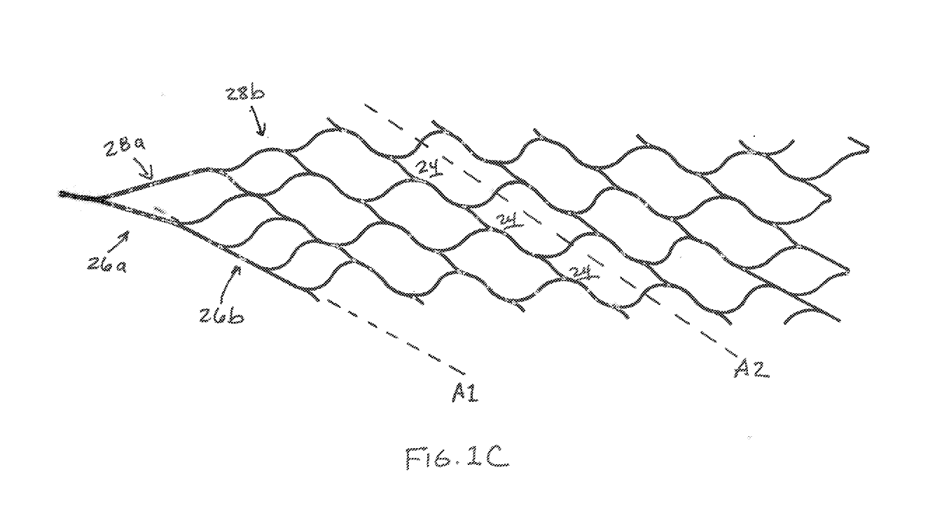

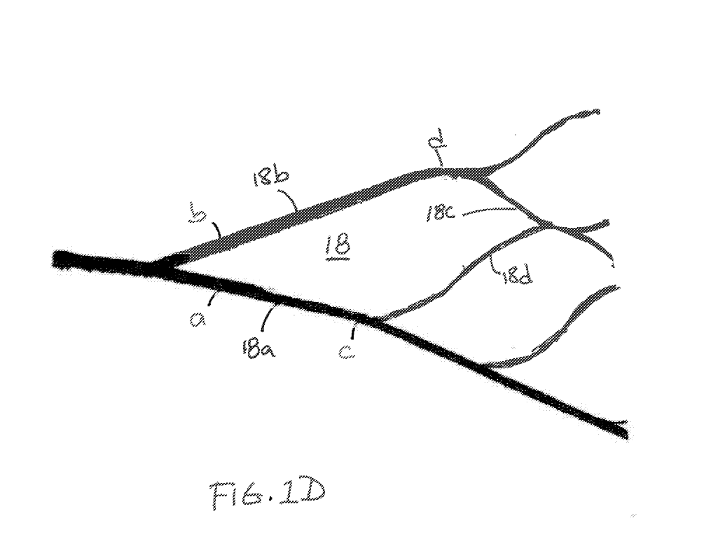

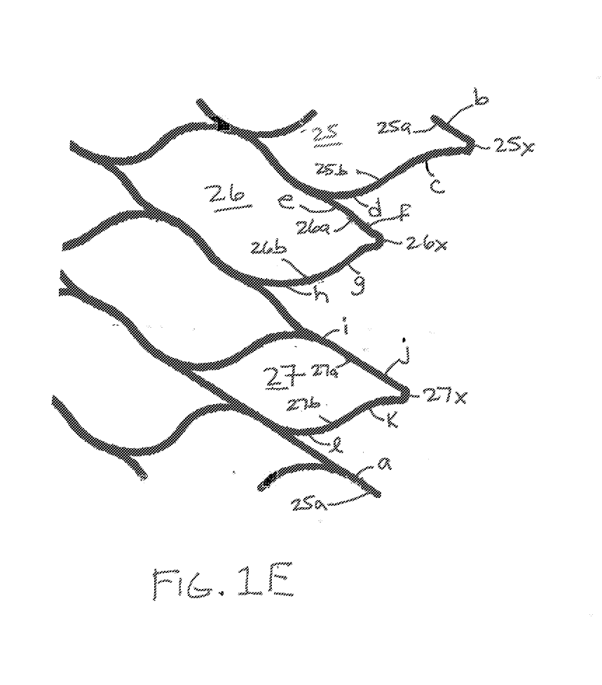

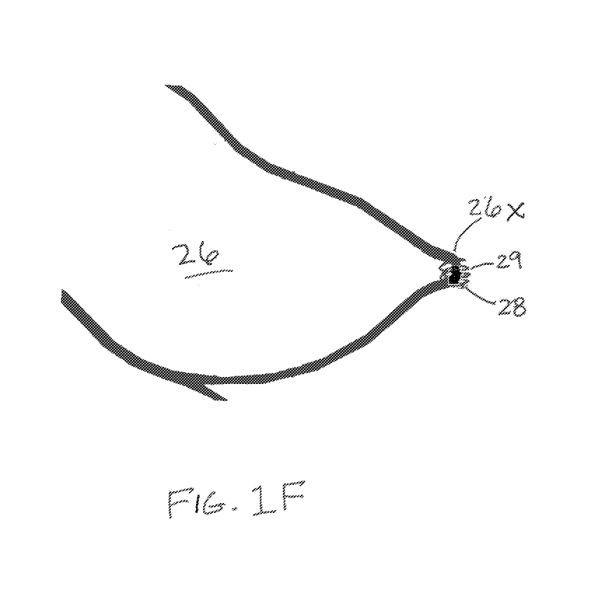

FIGS. 1A-1C illustrate a vascular or bodily duct treatment device 10 in accordance with one implementation. The device illustrated in FIG. 1C is the same as in FIG. 1A, the difference lying in the annotation of the figure. Device 10 is particularly suited for accessing and treating the intracranial vascular of a patient, such as for example treating aneurysms or capturing and removing embolic obstructions. It is appreciated however, that device 10 may be used for accessing and treating other locations within the vasculature and also other bodily ducts. Other uses include, for example, treating stenoses and other types of vascular diseases and abnormalities. FIG. 1A depicts device 10 in a two-dimensional plane view as if the device were cut and laid flat on a surface. FIG. 1B depicts the device in its manufactured and/or expanded tubular configuration. Device 10 includes a self-expandable member that includes a main body portion 12, a proximal taper portion 14 and a distal portion 16. The main body portion 12 includes a plurality of cell structures that are arranged to form a substantially cylindrical tubular structure as shown in FIG. 1B, the cell structures in the main body portion extending continuously and circumferentially around a longitudinal axis of the expandable member. According to some implementations the cell structures 24 in the main body portion 12 are arranged so that no cell structure therein is circumferentially aligned with any adjacent cell structure as best shown in FIG. 1A. With respect to the implementation of FIGS. 1A and 1B, each row of cell structures 24 in the main body portion 12 circumscribes the device in a diagonal fashion with respect to the longitudinal axis that extends through the center of the expandable member. The cell structures 24 labeled in FIG. 1A represent a row of cell structures and represent the diagonal disposition of the cells. The proximal taper portion 14 includes a plurality of cell structures extending less than circumferentially around the longitudinal axis of the expandable member as best shown in FIG. 1B. According to one implementation the expandable member is made of shape memory material, such as Nitinol, and is preferably laser cut from a tube. According to some implementation the expandable member has an integrally formed proximally extending antenna 30 that is used to join a proximally extending elongate flexible wire (not shown) to the expandable member.

In use, a proximal end of the flexible wire resides outside the body of the patient and is manipulated by the physician to navigate and push the device 10 through the anatomy of the patient. The flexible wire may be joined to the antenna 30 by the use of solder, a weld, an adhesive, or other known attachment methods. In other implementations the antenna 30 is omitted and the distal end of the flexible wire is attached directly to a proximal end 17 of the expandable member. In use, the expandable member is delivered to the treatment site of a patient through a delivery catheter that is pre-positioned at or proximal to the treatment site. The expandable member may be deployed at the treatment site by advancing the expandable member distally until it emerges from the distal end of the delivery catheter and/or by a proximal retraction of the delivery catheter. As will be discussed in more detail below, the expandable member of device 10 comprises a variety of features that enhance its ability to be reintroduced (re-sheathed) into the delivery catheter. Because misplacement of the expandable member inside the patient is possible, the ability to reintroduce the expandable member into the delivery catheter and to alter the deployment location is important.

In use, the expandable member is advanced through the tortuous vascular anatomy or bodily duct of a patient to a treatment site in an unexpanded or compressed state (not shown) of a first nominal diameter and is expandable from the unexpanded state to a radially expanded state of a second nominal diameter greater than the first nominal diameter for deployment at the treatment site. In one implementation, the dimensional and material characteristics of the cell structures 24 residing in the main body portion 12 of the expandable member are selected to produce sufficient radial force and contact interaction to cause the cell structures 24 to engage with an embolic obstruction residing in the vascular in a manner that permits partial or full removal of the embolic obstruction from the patient. In alternative embodiments the dimensional and material characteristics of the cell structures 24 in the main body portion 12 are selected to produce a radial force per unit length of between about zero N/mm to about 0.050 N/mm, preferable between about 0.010 N/mm to about 0.050 N/mm, and more preferably between about 0.030 N/mm and about 0.050 N/mm. In one embodiment, the diameter of the main body portion 12 in a fully expanded state is about 4.0 millimeters with the cell pattern, strut dimensions and material being selected to produce a radial force of between about 0.040 N/mm to about 0.050 N/mm when the diameter of the main body portion is reduced to between about 1.0 millimeters to about 1.5 millimeters. In the same or alternative embodiment, the cell pattern, strut dimensions and material(s) are selected to produce a radial force of between about 0.010 N/mm to about 0.020 N/mm when the diameter of the main body portion is reduced to 3.0 millimeters.

According to some implementations a majority of the cell structures 24 in the main body portion 12 are constructed by the interconnection of out-of-phase undulating elements 25a-d that extend along a length of the expandable member. This construction provides a number of advantages. First, the curvilinear nature of the cell structures 24 enhances the flexibility of the expandable member during its delivery through the tortuous anatomy of the patient to the treatment site. In addition, the out-of-phase relationship between the undulating elements 25a-d facilitates a more compact nesting of the expandable member elements permitting the expandable member to achieve a very small compressed diameter. A particular advantage of the expandable member strut pattern shown in FIG. 1A, and various other implementations described herein, is that they enable sequential nesting of the expandable member elements which permit the expandable members to be partially or fully deployed and subsequently withdrawn into the lumen of a delivery catheter. The out-of-phase relationship also results in a diagonal orientation of the cell structures 24 which may induce a twisting action as the expandable member transitions between the compressed state and the expanded state that helps the expandable member to better engage with the embolic obstruction.

According to one implementation in an as-cut manufactured state the expandable member has an overall length of about 32.0 millimeters with the main body portion 12 having a length L2 of about 18.0 millimeters, the proximal taper portion 14 having a length L1 of about 10.0 mm and the distal portion 16 having a length L3 of about 4.0 millimeters. According to another implementation in an as-cut manufactured state the expandable member has an overall length of about 36.0 millimeters with the main body portion 12 having a length L2 of about 21.0 millimeters, the proximal taper portion 14 having a length L1 of about 11.0 mm and the distal portion 16 having a length L3 of about 4.0 millimeters. According to some implementations the ratio of the length L1 of the proximal taper portion 14 with the length L4 of the proximal-most cell 18 is between about 1.4 and about 2.0. That is, L1/L4 is between about 1.4 and about 2.0. According to some implementations the length of the proximal-most cell is between about 6.0 and 7.0 millimeters. As a result of the length of the proximal-most cell structure 18 being relatively long in comparison to the length of the proximal end portion 14, the angle α may be advantageously minimized to reduce the amount of overall force required to reintroduce the expandable member into the delivery catheter in the event the expandable member is improperly positioned as discussed above. During an initial sheathing of the expandable member into the delivery catheter the smaller angle also prevents high normal forces from occurring in the delivery catheter, thus resulting in less resistance as the expandable member is advanced through the catheter. Another advantage is that foreshortening contributed by the proximal-most cell structure 18 is minimized as the expandable member expands from a compressed state when housed in the delivery catheter to an expanded state when the expandable member is deployed outside the delivery catheter. According to some implementations the angle α is between about 20 and about 25 degrees. According to other implementations the angle α is between about 20 and about 30 degrees. According to other implementations the angle α is between about 20 and about 40 degrees.

With continued reference to FIGS. 1A-1C, the proximal taper portion 14 is delimited by first and second rail segments 26 and 28, respectively, with each rail segment extending from the proximal end 17 of the expandable member to the main body portion 12 of the device. The first rail segment 26 is defined by the outer-most struts 18a, 21a, 22a and 23a of cell structures 18, 21, 22 and 23, respectively. The second rail segment 28 is defined by the outer-most struts 18b, 19a and at least a portion of 20a of cell structures 18, 19 and 20, respectively. The first rail segment 26 comprises a first linear or substantially linear portion 26a defined at least in part by the proximal portion of strut 18a and a second linear or substantially linear portion 26b defined by struts 21a, 22a and 23a, with the angular orientation of the second portion 26b being different from the angular orientation of the first portion 26a. As shown in FIGS. 1A and 1C, when the expandable member is cut and laid flat on a surface the second portion 26b of rail segment 26 has an angular orientation that is different than the angular orientation of the first portion 26a. As a result of the relatively long length of the proximal-most cell structure 18 in comparison to the length of the proximal end portion 14, the divergence in angular orientation between the first and second portions 26a and 26b facilitates a shorter proximal end portion 14 length than would otherwise be achievable if the angular orientation of the second portion 26b remained the same as the angular orientation of the first portion 26a of rail segment 26. In order to enhance the expandable member's ability to collapse and to facilitate a nesting among the cell structures that form it, according to some implementations the second portion 26b of rail segment 26 has an angular orientation similar to the helix angle followed by a majority of the cell structures 24 in the main body portion 12 of the device. As shown in FIG. 1C the line A1 coextending from the second portion 26b of rail segment 26 has an angular orientation similar to line A2 which represents the helix angle when the expandable member is cut and laid flat on a surface. According to some implementations the difference in angular orientation of lines A1 and A2 is between zero and 10 degrees and preferably between zero and 6 degrees.

The second rail segment 28 comprises a linear portion 28a and an undulating portion 28b, the linear portion 28a being defined at least in part by the proximal portion of strut 18b. According to some implementations the struts 18b, 19a, 20a of rail segment 28 and the struts 18a, 21a, 22a, 23a of rail segment 26 are constructed so that the length of the rail segments 26 and 28 are similar when the expandable member is in a compressed configuration when housed within the delivery catheter. By minimizing the mismatch in length between rail segments 26 and 28 the cell structures in the proximal end portion 14 of the expandable member more readily nest and the formation of bulges and other irregularities in profile are minimized. According to some implementations the difference in length between the first rail segment 26 and the second rail segment 28 is no greater than 2%. According to other implementations the difference in length between the first rail segment 26 and the second rail segment 28 is no greater than 5%.

FIG. 1D depicts an enlarged view of the proximal-most cell structure 18 according to some implementations. The proximal end portions of struts 18a and 18b that originate at the proximal end 17 of the expandable member are straight or substantially straight when the expandable member is cut and laid flat on a surface as shown in FIG. 1D. The linear or straight nature of the proximal end portions of struts 18a and 18b provide good column strength that resists against bending and/or buckling as the expandable member is pushed through the delivery catheter. The proximal end portions of struts 18a and 18b along locations “a” and “b” have a greater width dimension than the width dimension at locations “c” and “d”, respectively. In some instances the width dimension of each of the proximal end portions of struts 18a and 18b are greater than the width dimension of all of the remaining struts or strut portions in the proximal end portion 14 of the expandable member. In some instances the width dimension of each of the proximal end portions of struts 18a and 18b are greater than the width dimension of all of the remaining struts or strut portions in the expandable member. By providing the proximal end portions of struts 18a and 18b with an enhanced width dimension good column strength is provided that resists against bending and/or buckling as the expandable member is pushed through the delivery catheter. According to some implementations the width dimension of the struts 18a and 18b at locations “a” and “b” is between about 0.0055 and 0.0060 inches with the width dimension at location “c” being between about 0.0049 and 0.0052 inches and the width dimension at location “d” being between about 0.0036 and 0.0045 inches. According to some implementations the width dimension of strut 18c may be in the range of about 0.0030 to 0.0033 inches and the width dimension of strut 18d may be in the range of about 0.0028 to 0.0030 inches.

According to some implementations the cell structures 24 that form the main body portion 12 of the expandable member are made of struts having a width dimension of between about 0.0025 to 0.0030 inches. According to some implementations each of the struts in the expandable member have substantially the same thickness dimension while in other implementations the thickness dimension of the struts vary. According to some implementations the thickness dimension is between about 0.0030 and 0.0035 inches.

FIG. 1E shows an enlarged view of the distal portion 16 of the expandable member depicted in FIG. 1A. The cell structures or portions of cell structures that form the distal portion 16 are constructed and arranged so that the expandable member comprises a substantially blunt or blunt-like end with the ends 25x, 26x and 27x of cell structures 25, 26 and 27 lying in parallel planes that are orthogonal to the longitudinal axis of the expandable member. In order to enhance the atraumatic quality of distal portion 16, the distal-most struts of cell structures 25, 26 and 27 are provided with regions of reduced width and/or thickness that allows the distal region of each of cell structures 25, 26 and 27 to flex. According to some implementations the regions of reduced width and/or thickness reside at or near the proximal end of the distal-most struts adjacent an intersection with a neighboring strut. By way of example and with continued reference to FIG. 1E, each of cell structures 25, 26 and 27 comprise pairs of distal-most struts 25a, 25b; 26a, 26b and 27a, 27b, respectively. Distal-most strut 25a comprises a proximal region “a” and a distal region “b” with the proximal region “a” having a width and/or thickness dimension less than that in distal region “b”. Distal-most strut 25b comprises a proximal region “d” and a distal region “c” with the proximal region “d” having a width and/or thickness dimension less than that in distal region “c”. As a result of this construction the distal region of cell structure 25 is permitted to flex with bending predominately occurring at the locations of reduced width and/or thickness, namely at locations “a” and “d”. Distal-most strut 26a comprises a proximal region “e” and a distal region “f” with the proximal region “e” having a width and/or thickness dimension less than that in distal region “f”. Distal-most strut 26b comprises a proximal region “h” and a distal region “g” with the proximal region “h” having a width and/or thickness dimension less than that in distal region “g”. As a result of this construction the distal region of cell structure 26 is permitted to flex with bending predominately occurring at the locations of reduced width and/or thickness, namely at locations “e” and “h”. Distal-most strut 27a comprises a proximal region “i” and a distal region “j” with the proximal region “i” having a width and/or thickness dimension less than that in distal region “j”. Distal-most strut 27b comprises a proximal region “l” and a distal region “k” with the proximal region “l” having a width and/or thickness dimension less than that in distal region “k”. As a result of this construction the distal region of cell structure 27 is permitted to flex with bending predominately occurring at the locations of reduced width and/or thickness, namely at locations “i” and “l”.

As shown in FIG. 1E, the distal ends 25x, 26x and 27x of cell structures 25, 26 and 27, respectively, comprise a nipple 28 suitable for receiving a radiopaque marker 29 in the form of a spiral structure that may be wound about a section of the nipple as illustrated in FIG. 1F. Other non-spiral types of radiopaque markers may also be affixed to the nipple region of one or more of cell structures 25, 26 and 27 by means bonding (e.g., solder, weld, epoxy, etc.), swaging or crimping. According to some implementations the inner width of the nipple is between about 0.006 and 0.007 inches and the strut onto which the radiopaque marker is attached has a width between about 0.0035 to 0.0045 inches.

According to some implementations the distal region of one or more of cell structures 25, 26 and 27 is formed to flare outward when the distal portion 16 of the expandable member resides outside the delivery catheter. As a result of being flared outward toward the vessel wall, the distal regions of cell structures 25, 26 and 27 may gently ride along the vessel wall upon the expandable member being withdrawn proximally through the vasculature of the patient and may collect residual debris resulting from the proximal movement of the treatment device 10 as it is being removed from the patient.

In the foregoing discussion of FIGS. 1A-1F numerous features have been described that both individually and collectively enhance the expandable member's ability to perform its function, which in some instances involves the delivery of the expandable member to a treatment site within a patient via a delivery catheter, the deployment of the expandable member at the treatment site into a vascular obstruction (e.g. blood clot), and the withdrawal of the expandable member from the patient along with the captured vascular obstruction. As previously discussed, placement of the expandable member at the treatment site can involve reintroducing or re-sheathing the expandable member into the delivery catheter. Placement can also involve proximal and distal movement of the expandable member inside the patient's vessel. With respect to each of these functions particular features or groups of features have been provided to enhance the expandable member's performance with respect to the function. As such, it is appreciated that a combination of all the features described herein with respect to the disclosed embodiments is not required and that each of the features may individually be implemented into an expandable member without a requirement to include the other disclosed features. Moreover, it is appreciated that any individual feature may be combined with one or more of the other features.

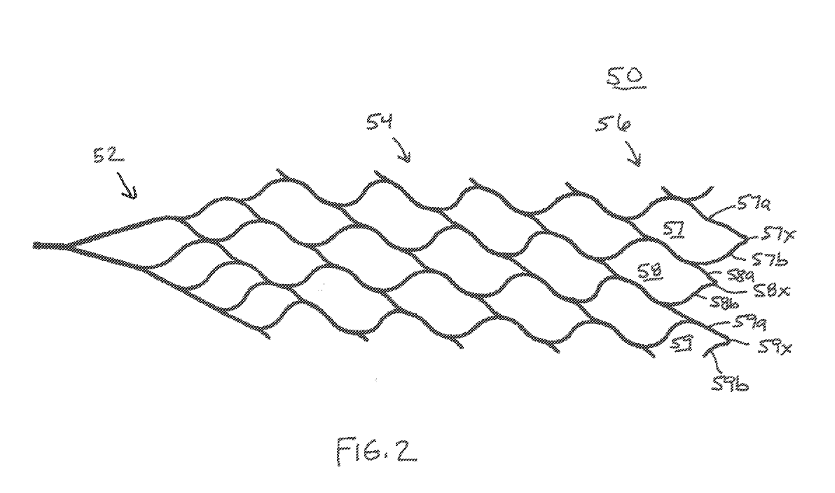

FIG. 2 illustrates a vascular or bodily duct treatment device 50 according to another implementation that is similar to the treatment device 10 depicted in FIG. 1A. The expandable member of device 50 has a proximal taper portion 52, a cylindrical main body portion 54 and a distal portion 56. Apart from dimensional differences that may occur between the expandable members of treatment devices 10 and 50, a difference lies in the construction of the distal portions 18 and 56. Like the expandable member of device 10, each of the distal-most cell structures 57, 58 and 59 of device 50 comprises a pair of distal-most struts 57a, 57b; 58a, 58b and 59a, 59b, respectively, with at least one or more of the distal-most struts comprising a proximal region and a distal region with the proximal region having a width and/or thickness dimension less than that in the distal region. As a result of this construction the distal region of one or more of the distal-most cell structures 57, 58 and 59 is permitted to flex with bending occurring predominately at the locations of reduced width and/or thickness.

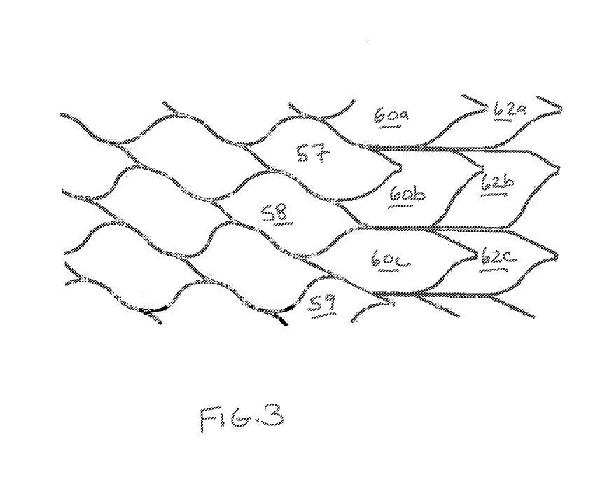

In the implementations illustrated in FIGS. 1A and 2, the distal ends of the distal-most cell structures within the distal portions 14 and 56 are staggered (i.e. do not lie within the same plane. According to some implementations in order to provide the expandable member with end points that lie substantially in the same plane one or more rows of cell structures may be added in a manner as illustrated in FIG. 3. By way of example, FIG. 3 illustrates the expandable member of FIG. 2 with two rows of cell structures appended to the end of the expandable member. It is important to note that a single row of cell structures may also be used. A first row of cell structures 60a, 60b and 60c extend distally from cell structures 57, 58 and 59 with their distal ends being substantially aligned in the same plane. A second row of cell structures 62a, 62b and 62c may also be provided. According to some implementations circumferentially adjacent cell structures are not attached to each other as shown in FIG. 3. That is, cell structure 60a is not connected to cell structure 60b, cell structure 60b is not connected to cell structure 60c, cell structure 60c is not connected to cell structure 60a, cell structure 62a is not connected to cell structure 62b, cell structure 62b is not connected to cell structure 62c and cell structure 62c is not connected to cell structure 62a. This form of construction endows the distal portion of the expandable member with greater flexibility. According to some implementations the distal portion of the expandable member that includes cell structures 60a-c and 62a-c may be formed to flare outward so as to have a greater expanded diameter than the main body portion. As a result of being flared outward toward the vessel wall, at least one or more portions of cell structures 60a-c and 62a-c may gently ride along the vessel wall upon the expandable member being withdrawn proximally through the vasculature of the patient and may collect residual debris resulting from the proximal movement of the treatment device 50 as it is being removed from the patient. According to some implementations the distal-most cell structures are flared outward in a linear manner with there being a single longitudinal location on the expandable member whereby cell structures 60a-c are bent. According to other implementations the distal-most cell structures are flared outward in a curvilinear manner with there being multiple bending locations so as to create, for example, a flare having a concave shape.

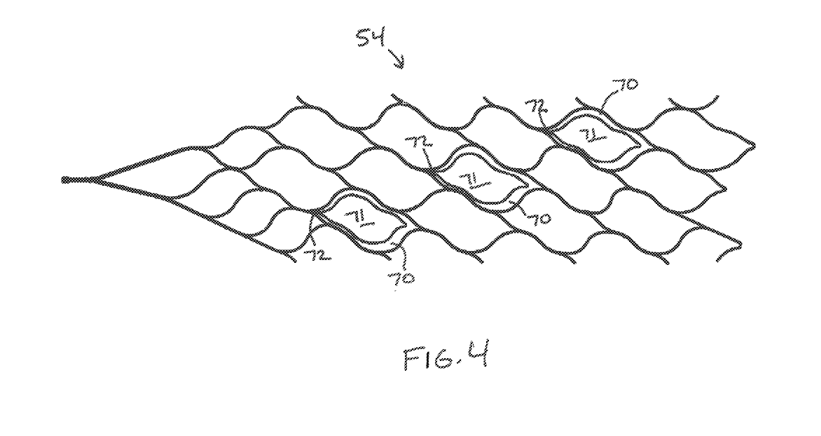

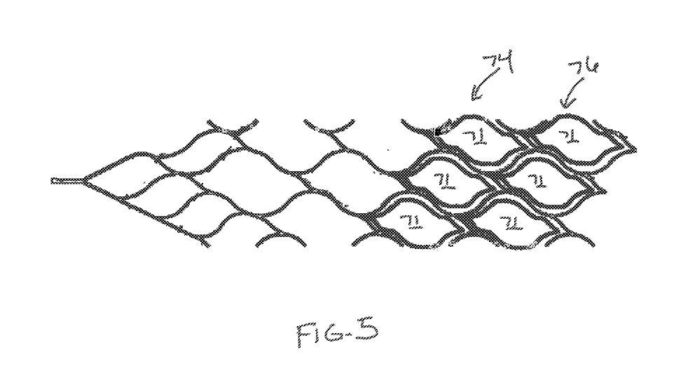

FIG. 4 illustrates a variant to the treatment device 50 shown in FIG. 2 with at least some of the cells 70 within the main body portion 54 comprising a floating inner cell 71. According to some implementations the inner cells 71 are shaped similarly to the cells 70 in which they are disposed and are attached at their proximal ends to the proximal apex 72 of cells 70. Other shapes and other attachment locations are also possible. According to some implementations the inner cells are biased to bend inward toward the center of the expandable member and function to assist in capturing debris within the expandable member and to inhibit the debris from escaping from the expandable member as it is moved within the vasculature or other bodily duct of the patient. According to some implementations the width of the struts that form cells 71 are smaller than the width of the struts that form cells 70. In other implementations one or more of rows of cells 74 and 76 located in the distal region of the expandable member comprise a floating inner cell 71 as shown in FIG. 5.

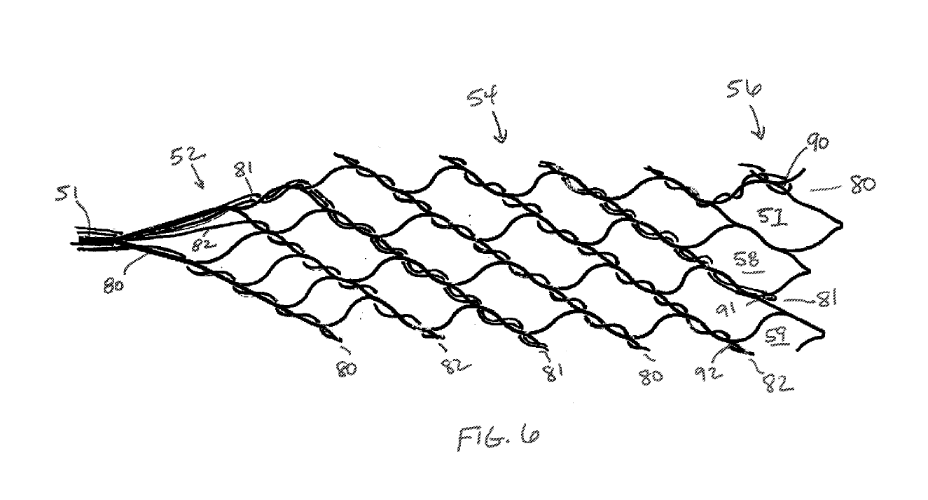

FIG. 6 illustrates an expandable member of a treatment device similar to that shown in FIG. 2 with there being provided a plurality of radiopaque wires wound about portions of the expandable member. In the embodiment of FIG. 6 there are provided three radiopaque wires 80, 81 and 82 that are wound about selective struts for the purpose of enhancing the radiopacity of the expandable member and/or to affect the stiffness of one or more portions of the device. In the exemplary implementation of FIG. 6 three radiopaque wires (or ribbons) 80, 81 and 82 are woven along a length of the retrieval device to enhance the radiopacity of the device along its length and to at least enhance the stiffness of at least the cylindrical main body portion 54. In order to avoid an undue stiffening of the distal portion 56 of the device, a majority or the entire distal portion 56 is devoid of wires 80-82. In the implementation of FIG. 6 the wires 80-82 are woven about the diagonally downward oriented struts (as viewed from left to right). Wrapping of each of the wires may be accomplished by folding the wire somewhere along its length, such as, for example, half way along its length, and positioning the fold at a distal location 90, 91 or 92, and then weaving the free ends along the struts as shown in FIG. 6. An advantage of this method is that no bonding of the wires 80, 81 and 82 at locations 90, 91 and 92, respectively, is required which would add unwanted bulk and stiffness to the distal portion 56. In one implementation the wires 80-82 comprise platinum with a width and/or diameter of between about 0.0015 inches and 0.0025 inches with there being an average of about one to ten windings per strut, and most generally one to five windings per strut. It is to be appreciated that a single wire or any multiple thereof may be used in lieu of the three wire configurations depicted in FIG. 6. Moreover, it is important to note that, in the case of enhancing radiopacity that the wire or wires may comprise any radiopaque material or combination of materials. In the event that wire windings are applied only for the purpose of affecting stiffness, the wire or wires may comprise any material suitable for such purpose, such as for example metallic, polymeric and composite materials. In some implementations the cross-sectional area of the one or more wires varies to provide a variation in radiopacity and/or stiffness along the length of the device. According to some implementations the proximal and distal end segments of wires 80-82 are coupled to the proximal antenna 51 of the expandable member. In some implementation, the ends of the wires 80-82 and the proximal antenna 51 are coupled together within a coil structure (not shown). In such an implementation the ends of wires 80-82 are interposed between the proximal antenna 51 and the coil that surrounds it. In such an implementation a bonding agent may be introduced into the interior of the coil to effectuate a bonding together the coil, proximal antenna 51 and the end of wires 80-82. In other implementations the ends of wires 80-82 are bonded directly to the proximal antenna 51 by use of a bonding agent such as solder or glue.

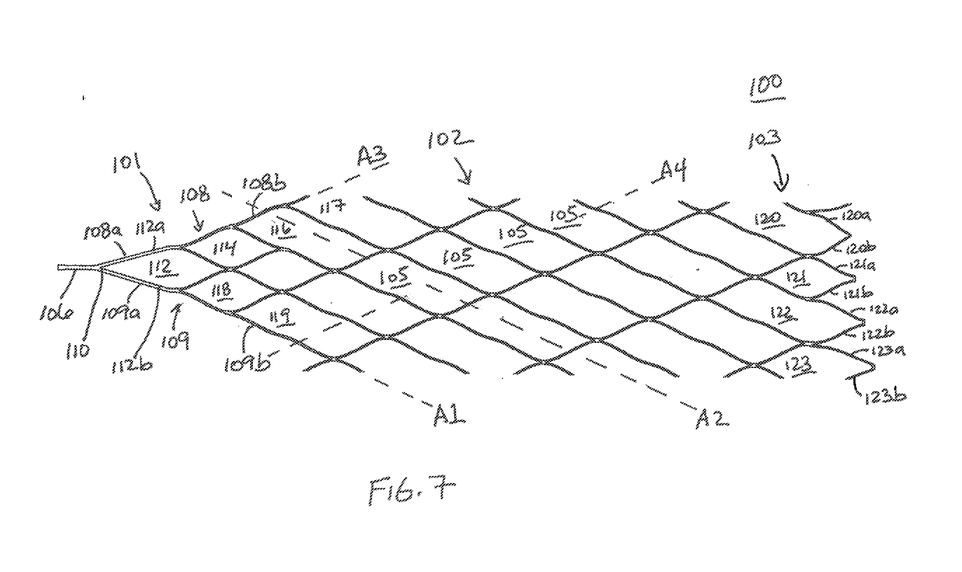

FIG. 7 illustrates a vascular or bodily duct treatment device 100 in accordance with one implementation with substantially all of the cells having a substantially parallelogram shape. FIG. 7 depicts device 100 in a two-dimensional plane view as if the device were cut and laid flat on a surface. Device 100 includes a self-expandable member that includes a proximal taper portion 101, a main body portion 102 and a distal portion 103. The main body portion 102 includes a plurality of cell structures that are arranged to form a substantially cylindrical tubular structure, the cell structures 105 in the main body portion extending continuously and circumferentially around a longitudinal axis of the expandable member. According to some implementations the cell structures 105 in the main body portion 102 are arranged so that no cell structure therein is circumferentially aligned with any adjacent cell structure as shown in FIG. 7. With respect to the implementation of FIG. 7, each row of cell structures 105 in the main body portion 102 circumscribes the device in a diagonal fashion with respect to the longitudinal axis that extends through the center of the expandable member. The cell structures 105 labeled in FIG. 7 represent a row of cell structures and represent a diagonal disposition of the cells. The proximal taper portion 101 includes a plurality of cell structures extending less than circumferentially around the longitudinal axis of the expandable member. According to one implementation the expandable member is made of shape memory material, such as Nitinol, and is preferably laser cut from a tube. According to some implementation the expandable member has an integrally formed proximally extending antenna 106 that is used to join a proximally extending elongate flexible wire (not shown) to the expandable member.

With continued reference to FIG. 7, the proximal taper portion 101 is delimited by first and second rail segments 108 and 109, respectively, with each rail segment extending from the proximal end 110 of the expandable member to the main body portion 102 of the device. The first rail segment 108 is defined by the outer-most struts of cell structures 112, 114, 116 and 117. The second rail segment 109 is defined by the outer-most struts of cell structures 112, 118 and 119. The first rail segment 108 comprises a first substantially linear portion 108a defined at least in part by the outer-most strut 112a of proximal-most cell structure 112. The first rail segment also comprises a second substantially linear portion 108b defined by the outer-most struts of cell structures struts 114, 116 and 117 with the angular orientation of the second substantially linear portion 108b being different from the angular orientation of the first substantially linear portion 108a. The second rail segment 109 comprises a first substantially linear portion 109a defined at least in part by the outer-most strut 112b of proximal-most cell structure 112. The first rail segment also comprises a second substantially linear portion 109b defined by the outer-most struts of cell structures struts 118 and 119 with the angular orientation of the second substantially linear portion 109b being different from the angular orientation of the first substantially linear portion 109a. The divergence in angular orientation between the first and second substantially linear portions of rail segments 108 and 109 facilitates a shorter proximal end portion 101 length than would otherwise be achievable if the angular orientation of the second substantially linear portions 108b and 109b remained the same as the angular orientation of the first substantially linear portions 108a and 108b, respectively. In order to enhance the expandable member's ability to collapse and to facilitate a nesting among the cell structures that form it, according to some implementations the second substantially linear portion 108b of rail segment 108 has an angular orientation A3 similar to a helix angle A4 followed by the cell structures 105 in the main body portion 102 of the device. As shown in FIG. 7 the line A3 coextending from the second substantially linear portion 108b of rail segment 108 has an angular orientation similar to line A4 which represents a helix angle when the expandable member is cut and laid flat on a surface. According to some implementations the angular orientation of line A3 is within zero and 5 degrees of the angular orientation of line A4. Likewise, according to some implementations the second substantially linear portion 109b of rail segment 109 has an angular orientation A1 similar to a helix angle A2 followed by the cell structures 105 in the main body portion 102 of the device. As shown in FIG. 7 the line A1 coextending from the second substantially linear portion 109b of rail segment 109 has an angular orientation similar to line A2 which represents a helix angle when the expandable member is cut and laid flat on a surface. According to some implementations the angular orientation of line A1 is within zero and 5 degrees of the angular orientation of line A2.

Like the expandable member of device 10 in FIG. 1A, each of the distal-most cell structures 120, 121, 122 and 123 of device 100 may comprise a pair of distal-most struts 120a, 120b; 121a, 121b; 122a, 122b and 123a, 123b, respectively, with at least one or more of the distal-most struts comprising a proximal region and a distal region with the proximal region having a width and/or thickness dimension less than that in the distal region. As a result of this construction the distal region of one or more of the distal-most cell structures 120, 121, 122 and 123 is permitted to flex with bending occurring predominately at the locations of reduced width and/or thickness.

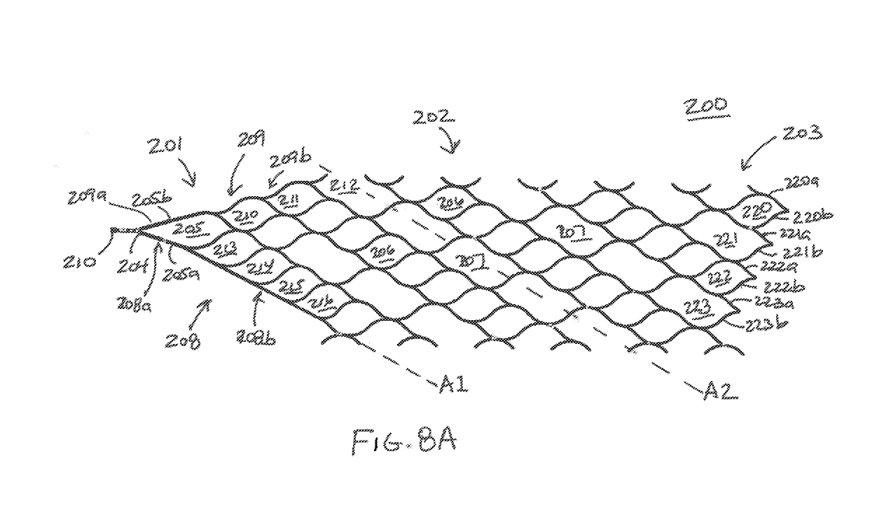

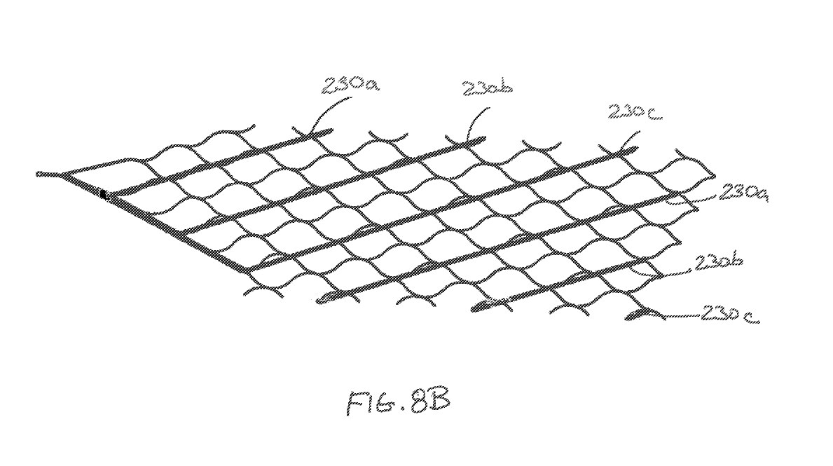

FIG. 8A illustrates a treatment device 200 according to another implementation. In FIG. 8A the treatment device is shown in a two-dimension plane view as if the device were cut and laid flat on a surface. The treatment device is a self-expandable member comprising a proximal taper portion 201, a main body portion 202 and a distal portion 203. The main body portion 202 includes a plurality of cell structures that are arranged to form a substantially cylindrical tubular structure with the cell structures in the main body portion extending continuously and circumferentially around a longitudinal axis of the expandable member. With respect to the implementation of FIG. 8A the main body of the expandable member comprises two types of cell structures 206 and 207 of different sizes. According to one implementation cell structures 206 are one half the size of cell structures 207 with each row of cell structures in the main body portion 202 circumscribing the device in a diagonal fashion with respect to the longitudinal axis that extends through the center of the expandable member. The proximal taper portion 201 includes a plurality of cell structures extending less than circumferentially around the longitudinal axis of the expandable member. According to one implementation the expandable member is made of shape memory material, such as Nitinol, and is preferably laser cut from a tube. According to some implementation the expandable member has an integrally formed proximally extending antenna 210 that is used to join a proximally extending elongate flexible wire (not shown) to the expandable member.

According to some implementations the proximal taper portion 201 is delimited by first and second rail segments 208 and 209, respectively, with each rail segment extending from the proximal end 204 of the expandable member to the main body portion 202 of the device. The first rail segment 208 is defined by the outer-most struts of cell structures 205, 213, 214, 215 and 216. The second rail segment 209 is defined by the outer-most struts of cell structures 205, 210, 211 and 212. The first rail segment 208 comprises a first linear portion 208a defined at least in part by the outer-most strut 205a of proximal-most cell structure 205 and a second linear portion 208b defined by the outer-most struts of cell structures 213-216, with the angular orientation of the second portion 208b being different from the angular orientation of the first portion 208a. As shown in FIG. 8A, when the expandable member is cut and laid flat on a surface the second portion 208b of rail segment 208 has an angular orientation that is different than the angular orientation of the first portion 208a. The divergence in angular orientation between the first and second portions 208a and 208b facilitates a shorter proximal end portion 201 length than would otherwise be achievable if the angular orientation of the second portion 208b remained the same as the angular orientation of the first portion 208a of rail segment 208. In order to enhance the expandable member's ability to collapse and to facilitate a nesting among the cell structures that form it, according to some implementations the second portion 208b of rail segment 208 has an angular orientation similar to the helix angle followed by the majority of the cell structures 206 and 207 in the main body portion 202 of the device. As shown in FIG. 8A the line A1 coextending from the second portion 208b of rail segment 208 has an angular orientation similar to line A2 which represents the helix angle of the cell structures in the main body portion when the expandable member is cut and laid flat on a surface. According to some implementations the angular orientation of line A1 is within ±5 degrees of the angular orientation of line A2. According to other implementations the angular orientation of line A1 is within ±10 degrees of the angular orientation of line A2.

The second rail segment 209 comprises a linear portion 209a and an undulating portion 209b. The linear portion 209a being defined at least in part by the outer-most strut 205b of proximal-most strut 205. The undulating portion 209b being defined at least in part by the outer-most struts of cell structures 210, 211 and 212. According to some implementations rail segments 208 and 209 have the same or substantially the same length when the expandable member is in a compressed configuration when housed within the delivery catheter. By minimizing the mismatch in length between rail segments 208 and 209 the cell structures in the proximal end portion 201 of the expandable member more readily nest and the formation of bulges and other irregularities in profile are minimized. According to some implementations the difference in length between the first rail segment 208 and the second rail segment 209 is no greater than 2%. According to other implementations the difference in length between the first rail segment 208 and the second rail segment 209 is no greater than 5%.

Like the expandable member of device 10 in FIG. 1A, each of the distal-most cell structures 220, 221, 222 and 223 of device 200 may comprise a pair of distal-most struts 220a, 220b; 221a, 221b; 222a, 222b and 223a, 223b, respectively, with at least one or more of the distal-most struts comprising a proximal region and a distal region with the proximal region having a width and/or thickness dimension less than that in the distal region. As a result of this construction the distal region of one or more of the distal-most cell structures 220, 221, 222 and 223 is permitted to flex with bending occurring predominately at the locations of reduced width and/or thickness.

FIG. 8B illustrates an expandable member of a treatment device similar to that shown in FIG. 8A with there being provided a plurality of wires wound about portions of the expandable member. According to some implementations the wires are radiopaque, In the embodiment of FIG. 8B there are provided a plurality of wires 230a-c that are wound about selective struts for the purpose of enhancing the radiopacity of the expandable member and/or to affect the stiffness of one or more portions of the device. In the exemplary implementation of FIG. 8B three radiopaque wires (or ribbons) 230a, 230b and 230c are woven along a length of the retrieval device to enhance the radiopacity of the device along its length and to at least enhance the stiffness of at least the cylindrical main body portion 202. In the implementation of FIG. 8B the wires 230a-c are woven about the diagonally upward oriented struts (as viewed from left to right) so that the wires bisect or substantially bisect the larger cell structures 207 in the cylindrical main body portion 202 of the device. This in effect increases the density of the cell structures in the main body portion 202 by augmenting the shape and size of the larger cell structures 207 in a manner to more resemble the shape and size of the smaller cell structures 206. This increases the radial force exerted by the main body portion 202 and provides the main body portion 202 with an outer wall surface having a more uniform distribution of open areas.

While the above description contains many specifics, those specifics should not be construed as limitations on the scope of the disclosure, but merely as exemplifications of preferred embodiments thereof. For example, dimensions other than those listed above are contemplated. For example, retrieval devices having expanded diameters of anywhere between 1.0 and 10.0 millimeters and lengths of up to 5.0 to 10.0 centimeters are contemplated. Moreover, it is appreciated that many of the features disclosed herein are interchangeable among the various implementations. Those skilled in the art will envision many other possible variations that are within the scope and spirit of the disclosure. Further, it is to be appreciated that the delivery of a vascular treatment device of the implementations disclosed herein is achievable with the use of a catheter, a sheath or any other device that is capable of carrying the device with the expandable member in a compressed state to the treatment site and which permits the subsequent deployment of the expandable member at a vascular treatment site. The vascular treatment site may be (1) at the neck of an aneurysm for diverting flow and/or facilitating the placement of coils or other like structures within the sack of an aneurysm, (2) at the site of an embolic obstruction with a purpose of removing the embolic obstruction, (3) at the site of a stenosis with a purpose of dilating the stenosis to increase blood flow through the vascular, etc.

What is claimed is:

1. A vascular or bodily duct treatment device comprising: an elongate self-expandable member having a radially expanded configuration and a radially unexpanded configuration, the self-expandable member comprising a plurality of cell structures, the self-expandable member having a proximal end portion and a substantially cylindrical main body portion disposed distal to the proximal end portion, the cell structures in the main body portion extending circumferentially around a longitudinal axis of the self-expandable member, the cell structures in the proximal end portion extending less than circumferentially around the longitudinal axis of the self-expandable member to form first and second peripheral rails, the first peripheral rail comprising a first substantially straight segment that originates at or near a proximal end of the self-expandable member and a second substantially straight segment that extends from the first substantially straight rail segment to a location at or near the substantially cylindrical main body portion, the angular orientation of the second substantially straight segment being different than the angular orientation of the first substantially straight segment, the angular orientation of the second substantially straight segment being similar to a helix angle of the cell structures in the main body portion.

2. A vascular or bodily duct treatment device comprising: an elongate self-expandable member having a radially expanded configuration and a radially unexpanded configuration, the self-expandable member comprising a plurality of cell structures, the self-expandable member having a proximal end portion, a substantially cylindrical main body portion disposed distal to the proximal end portion and a distal portion disposed distal to the substantially cylindrical main body portion, the cell structures in the substantially cylindrical main body portion and distal portion extending circumferentially around a longitudinal axis of the self-expandable member, the cell structures in the proximal end portion extending less than circumferentially around the longitudinal axis of the self-expandable member, the distal portion comprising a plurality of distal-most cell structures with each of the plurality of distal-most cell structures comprising a pair of distal-most struts that at least partially form an end segment of the respective distal-most cell structures, the distal-most struts comprising a proximal region and a distal region with at least some of the proximal regions having a width and/or thickness dimension less than the width and/or dimension of the respective distal regions.
